(19) United States
(12) Patent Application Publication
(10) Pub. No.: US 2025/0221623 A1
(43) Pub. Date: Jul. 10, 2025

(54) INTRAVASCULAR IMAGING CATHETER

(71) Applicant: Boston Scientific Scimed, Inc., Maple Grove, MN (US)

(72) Inventors: Andrew Strom, Golden Valley, MN (US); Quinn Mackenzie Butler, Minneapolis, MN (US); Bryan Joseph Plunger, Shoreview, MN (US); Jeffrey Steven Fuller, Brooklyn Park, MN (US); Monica Ortiz Serrano, Maple Grove, MN (US); Laura Quesada Soto, Alajuela (CR); Brian Chavarria, Maple Grove, MN (US)

(73) Assignee: Boston Scientific Scimed, Inc., Maple Grove, MN (US)

(21) Appl. No.: 19/011,884

(22) Filed: Jan. 7, 2025

Related U.S. Application Data
(60) Provisional application No. 63/618,555, filed on Jan. 8, 2024.

(51) Int. Cl. A61B 5/00; A61B 90/00

(52) U.S. Cl. CPC A61B 5/0066; A61B 5/6852; A61B 90/39; A61B 2090/3966

(57) ABSTRACT

Intravascular imaging devices as well as methods for making and using intravascular imaging devices are disclosed. An intravascular imaging device may include a catheter shaft including a proximal section and a distal section. The distal section may include a dual lumen region and a distal tip region coupled to the dual lumen region. The distal tip region may include a bumper tip member, a radiopaque member, and a sleeve disposed over and coupling the bumper tip member and the radiopaque member. An imaging core may be disposed within the catheter shaft.

### CROSS-REFERENCE TO RELATED APPLICATIONS

This application claims the benefit of priority under 35 U.S.C. § 119 of U.S. Provisional Application No. 63/618,555, filed Jan. 8, 2024, the entire disclosure of which is hereby incorporated by reference.

### TECHNICAL FIELD

The present disclosure pertains to medical devices, and methods for manufacturing medical devices. More particularly, the present disclosure pertains to intravascular imaging catheters.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An intravascular imaging device is disclosed. The intravascular imaging device comprises: a catheter shaft including a proximal section and a distal section; wherein the distal section includes a dual lumen region and a distal tip region coupled to the dual lumen region; wherein the distal tip region includes a bumper tip member, a radiopaque member, and a sleeve disposed over and coupling the bumper tip member and the radiopaque member; and an imaging core disposed within the catheter shaft.

Alternatively or additionally to any of the embodiments above, the imaging core is translatable within the catheter shaft.

Alternatively or additionally to any of the embodiments above, the imaging core includes an ultrasound transducer.

Alternatively or additionally to any of the embodiments above, the imaging core includes an optical coherence tomography imaging device.

Alternatively or additionally to any of the embodiments above, the dual lumen region has a distal end and further comprising a spacer member disposed between the radiopaque member and the distal end of the dual lumen region.

Alternatively or additionally to any of the embodiments above, the sleeve extends over at least a portion of the spacer member.

Alternatively or additionally to any of the embodiments above, the sleeve is thermally bonded to the dual lumen region.

Alternatively or additionally to any of the embodiments above, the dual lumen region includes a guidewire lumen and an imaging core lumen.

Alternatively or additionally to any of the embodiments above, the dual lumen region has a skived opening in fluid communication with the guidewire lumen.

Alternatively or additionally to any of the embodiments above, an imaging window region is coupled to the dual lumen region adjacent to the imaging core lumen.

Alternatively or additionally to any of the embodiments above, a portion of dual lumen region adjacent to the imaging core lumen is disposed along an outer surface of the imaging window region.

Alternatively or additionally to any of the embodiments above, the imaging window region has a distal portion having a first outer diameter and a proximal portion having a second outer diameter larger than the first outer diameter.

An intravascular imaging device is disclosed. The intravascular imaging device comprises: a catheter shaft including a proximal hypotube section, an imaging window section having a lumen formed therein, and a distal section; wherein the distal section includes a dual lumen region and a distal tip region coupled to the dual lumen region; wherein the dual lumen region defines a guidewire lumen and defines an imaging core lumen in fluid communication with the lumen formed in the imaging window section; wherein the distal tip region includes a tip member, a radiopaque member, and a sleeve disposed over and coupling the tip member and the radiopaque member; and an imaging core disposed within the catheter shaft.

Alternatively or additionally to any of the embodiments above, the dual lumen region has a distal end and further comprising a spacer member disposed between the radiopaque member and the distal end of the dual lumen region.

Alternatively or additionally to any of the embodiments above, the sleeve extends over at least a portion of the spacer member.

Alternatively or additionally to any of the embodiments above, the sleeve is thermally bonded to the dual lumen region.

Alternatively or additionally to any of the embodiments above, the dual lumen region has a skived opening in fluid communication with the guidewire lumen.

Alternatively or additionally to any of the embodiments above, a portion of dual lumen region adjacent to the imaging core lumen is disposed along an outer surface of the imaging window section.

Alternatively or additionally to any of the embodiments above, the imaging window section has a distal portion having a first outer diameter and a proximal portion having a second outer diameter larger than the first outer diameter.

A method for imaging a blood vessel is disclosed. The method comprises: disposing an intravascular imaging device within a blood vessel, the intravascular imaging device comprising: a catheter shaft including a proximal section and a distal section, wherein the distal section includes a dual lumen region and a distal tip region coupled to the dual lumen region, wherein the distal tip region includes a bumper tip member, a radiopaque member, and a sleeve disposed over and coupling the bumper tip member and the radiopaque member, and an imaging core disposed within the catheter shaft; and translating the imaging core relative to the catheter shaft.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of a portion of an example medical device.

FIG. 2 is a side view of an example medical device.

FIG. 3 is a partial cross-sectional side view of a portion of an example medical device.

FIG. 4 is an exploded view of a portion of an example medical device.

FIG. 5 is a side view of a portion of an example medical device.

FIG. 6 is a cross-sectional view of a portion of an example medical device taken through line 6-6 in FIG. 4.

FIG. 7 is an alternative cross-sectional view of a portion of an example medical device.

FIG. 8 is a cross-sectional view of a portion of an example medical device taken through line 8-8 in FIG. 4.

FIG. 9 is an alternative cross-sectional view of a portion of an example medical device.

FIG. 10 is a side view of a portion of an example medical device.

FIG. 11 is a side view of a portion of an example medical device.

FIG. 12 is a side view of a portion of an example medical device.

FIG. 13 is a side view of a portion of an example medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

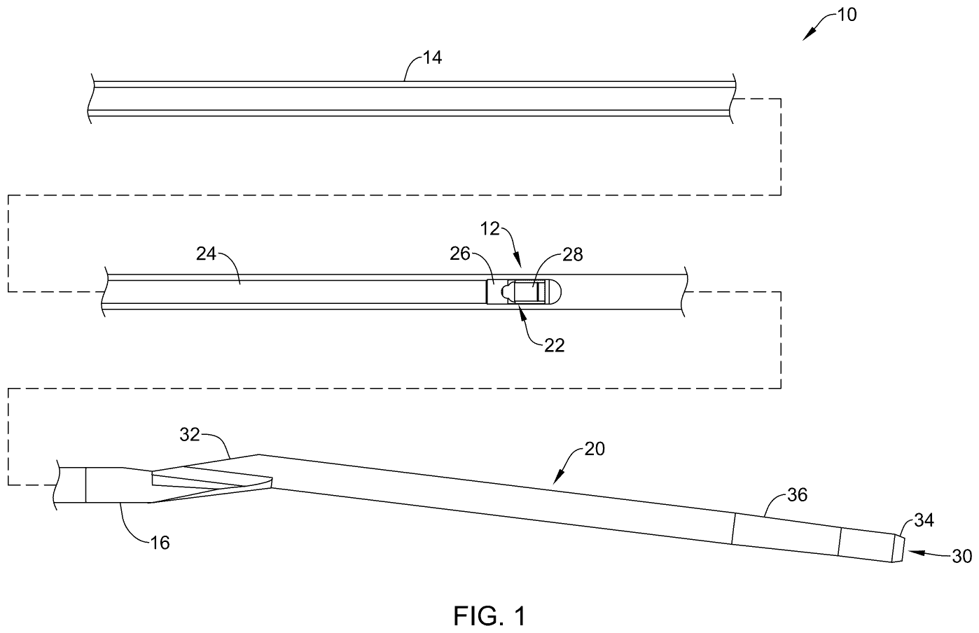

FIG. 1 is a side view of a portion of example medical device 10. In at least some instances, the medical device 10 takes the form of an imaging medical device. For example, the medical device 10 may be an intravascular ultrasound (IVUS) device that may be used to image a blood vessel. In some of these and in other instances, the medical device may be an optical coherence tomography (OCT) imaging device, a near-infrared spectroscopy (NIRS) imaging device, near-infrared fluorescence (NIRF) imaging device, a photoacoustic imaging device, a fluorescence-lifetime imaging device, combinations thereof, and/or the like. The structure/form of the medical device 10 can vary. In some instances, the medical device 10 may include an elongate shaft 12 having a proximal end region 14 and a distal end region 16. A tip member 20 may be coupled to or otherwise be disposed adjacent to the distal end region 16. The tip member 20 may include a guidewire lumen 30 having a guidewire exit port 32, an atraumatic distal end 34, one or more radiopaque markers 36, and/or other features. In some embodiments, the tip member 20 may extend at a non-parallel angle to the proximal end region 14 of the elongate shaft 12.

An imaging assembly 22 (e.g., which may sometime be referred to as an imaging core) may be disposed within a lumen of the elongate shaft 12. In general, the imaging assembly 22 may be used to capture/generate images of a blood vessel. In some instances, the medical device may include devices and/or features similar to those disclosed in U.S. Patent Application Pub. No. US 2012/0059241 and U.S. Patent Application Pub. No. US 2017/0164925, the entire disclosures of which are herein incorporated by reference. In at least some instances, the medical device 10 may resemble and/or include features that resemble the OPTICROSS™ Imaging Catheter, commercially available from BOSTON SCIENTIFIC, Marlborough, MA.

The imaging assembly 22 may include a drive shaft or cable 24, a housing 26, and an imaging member or transducer 28 coupled to the drive shaft 24 and/or housing 26. In at least some instances, the transducer 28 includes an ultrasound transducer. Other transducers are also contemplated. The transducer 28 may be rotatable and/or axially translatable relative to the elongate shaft 12. For example, the drive shaft 24 may be rotated and/or translated in order to rotate and/or translate the transducer 28 (and the housing 26).

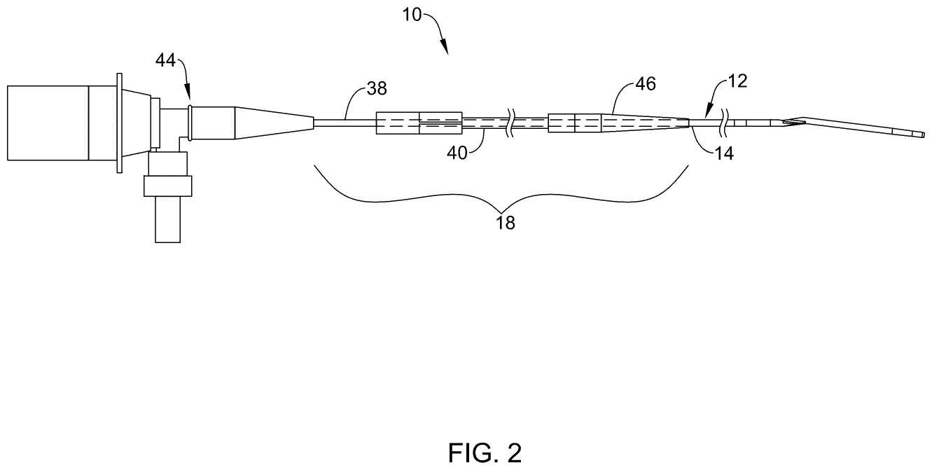

The proximal end region 14 of the elongate shaft 12 may be coupled to a telescoping assembly 18 as shown in FIG. 2. In general, the telescoping assembly 18 may be configured to allow the medical device operator to move the drive shaft 24 including the imaging assembly 22 proximally and distally within the catheter (e.g., relative to the elongate shaft 12), without having to move the entire catheter within the patient. This allows the catheter operator to easily change the location of the imaging assembly or other medical device within the patient. For example, the telescoping assembly 18 may be actuated to change the location of the imaging assembly 22 within the elongate shaft 12.

The proximal end region 14 of the elongate shaft 12 may be coupled to the telescoping assembly 18. For example, the proximal end region 14 of the elongate shaft 12 may be coupled to a distal hub 46 of the telescoping assembly 18. A proximal hub 44 may be coupled to the telescoping assembly 18 (e.g., at the proximal end of the telescoping assembly 18). The drive shaft 24 (see FIG. 1) may extend through the telescoping assembly 18 and be coupled to and/or otherwise secured to the proximal hub 44.

The telescoping assembly 18 may include a first sheath 38 and a second sheath 40. In some instances, the first sheath 38 may be understood to be an inner telescoping tube 38 and the second sheath 40 may be understood to be an outer telescoping tube 40. Generally, the outer telescoping tube 40 may be disposed over the inner telescoping tube 38. The inner telescoping tube 38 may be coupled to or otherwise secured to the proximal hub 44. The outer telescoping tube 40 may be coupled or otherwise secured to the distal hub 46. The inner telescoping tube 38 may be axially and/or rotatably moveable relative to the outer telescoping tube 40. Because the drive shaft 24 may be secured to the proximal hub 44 and/or the inner telescoping tube 38 and because the elongate shaft 12 may be secured to the distal hub 46, movement of the proximal hub 44 relative to the distal hub 46 results in movement of the inner telescoping tube 38 and the drive shaft 24 relative to the distal hub 46 and/or the elongate shaft 12.

In use, the elongate shaft 12 may be disposed within a target region (e.g., a blood vessel) and the imaging assembly 22 may be translated within the elongate shaft 12 in order to image the blood vessel. It can be appreciated that navigating the elongate shaft 12 through the vasculature toward the target region may include navigating the elongate shaft 12 through a number of tortuous bends and turns. As such, it may be desirable for the elongate shaft 12 to be sufficiently flexible in order to navigate such anatomy. Furthermore, it may be desirable for the elongate shaft 12 to be sufficiently pushable (e.g., in a manner that resists buckling) and be capable of transmitting torque along the length of the elongate shaft 12. Disclosed herein are medical devices (e.g., such as the medical device 10) where the elongate shaft 12 is designed to have a desirable level of flexibility, pushability, torquability, and/or other characteristics.

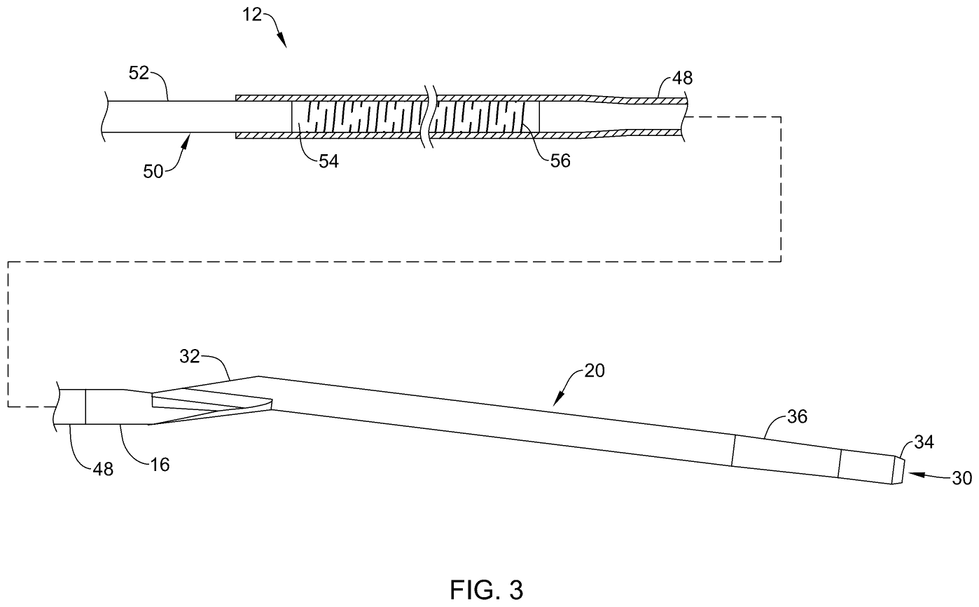

FIG. 3 illustrates a portion of the elongate shaft 12. As shown and described, the elongate shaft 12 may be an assembly of different parts/regions (e.g., the elongate shaft 12 may be understood to be a catheter shaft or catheter shaft assembly). For example, the elongate shaft 12 may include an imaging window region 48. As the name suggests, the imaging window region 48 is a region of the elongate shaft 12 through which the imaging assembly 22 (e.g., the transducer 28) can image through. While the imaging assembly 22 is not shown in FIG. 3, it can be appreciated that the imaging assembly 22 may be disposed within the elongate shaft 12 in the manner depicted in FIG. 1, for example. The imaging window region 48 may have a distal end that is coupled to and/or otherwise disposed adjacent to the tip member 20. In some instances, the imaging window region 48 extends the full length of the elongate shaft 12 (e.g., the full length proximally from the tip member 20). In other instances, the imaging window region 48 may extend along a portion of the elongate shaft 12. For example, the imaging window region 48 may have a length of about 5-50 cm (1.97-19.7 inches), or about 10-30 cm (3.94-11.8 inches), or about 15-25 cm (5.91-9.84 inches), or about 20-22 cm (7.87-8.66 inches). The imaging window region 48 may be formed from a suitable material such as nylon, nylon-12, polyether block amide, combinations thereof, and/or other suitable materials including those materials disclosed herein.

The elongate shaft 12 may also include a hypotube region 50. The hypotube region 50 may extend proximally from the imaging window region 48 to the distal hub 46. In some instances, the hypotube region 50 may be free of slots. In other instances, the hypotube region 50 may include a first portion 52 and a second portion 54. The first portion 52 may be free of slots. The second portion 54 may have a plurality of slots 56 formed therein. The slots 56 may help to provide a desirable level of flexibility (and/or pushability and/or torqueability) of the elongate shaft 12. Various arrangements and configurations are contemplated for slots 56. For example, in some embodiments, at least some, if not all of the slots 56 are disposed at the same or a similar angle with respect to the longitudinal axis of the hypotube region 50. In some instances, the slots 56 can be disposed at an angle that is perpendicular, or substantially perpendicular, and/or can be characterized as being disposed in a plane that is normal to the longitudinal axis of the hypotube region 50. However, in other instances, the slots 56 can be disposed at an angle that is not perpendicular, and/or can be characterized as being disposed in a plane that is not normal to the longitudinal axis of the hypotube region 50. This may include angled slots 56, slots 56 arranged in a spiral or helical pattern/arrangement, and/or the like. Additionally, a group of one or more slots 56 may be disposed at different angles relative to another group of one or more slots 56. The distribution and/or configuration of the slots 56 can also include, to the extent applicable, any of those disclosed in U.S. Pat. Publication No. US 2004/0181174, the entire disclosure of which is herein incorporated by reference.

The slots 56 can be formed by methods such as micro-machining, saw-cutting (e.g., using a diamond grit embedded semiconductor dicing blade), electrical discharge machining, grinding, milling, casting, molding, chemically etching or treating, or other known methods, and the like. In at least some embodiments, the slots 56 may be formed in tubular member using a laser cutting process. The laser cutting process may include a suitable laser and/or laser cutting apparatus. For example, the laser cutting process may utilize a fiber laser. In some embodiments, the slots 56 may have a width of about 0.005-0.04 mm (about 0.0002-0.0016 inches), or about 0.01-0.03 mm (about 0.0004-0.0012 inches), or about 0.02 mm (about 0.0079 inches). The width of the slots 56 may be constant or may be vary along the length of the hypotube region 50. The slots 56 may have an arc length of about 35-75 degrees, or about 40-60 degrees, or about 50 degrees. The spacing between axially-adjacent slots 56 may be about 0.1-4 mm (about 0.0004-0.16 inches), or about 0.2-3 mm (about 0.0008-0.12 inches), or about 0.25-2 mm (about 0.01-0.08 inches). The spacing may be constant or may vary along the length of the hypotube region 50.

During a coronary intervention, an imaging device may be navigated through the tortuous anatomy. When doing so, it is possible that the imaging device may kink or otherwise deform in a way that may disrupt the function of the device. Disclosed herein are intravascular imaging devices that include structural features to the device and that, for example, may help to reduce kinking as well as provide additional desirable benefits.

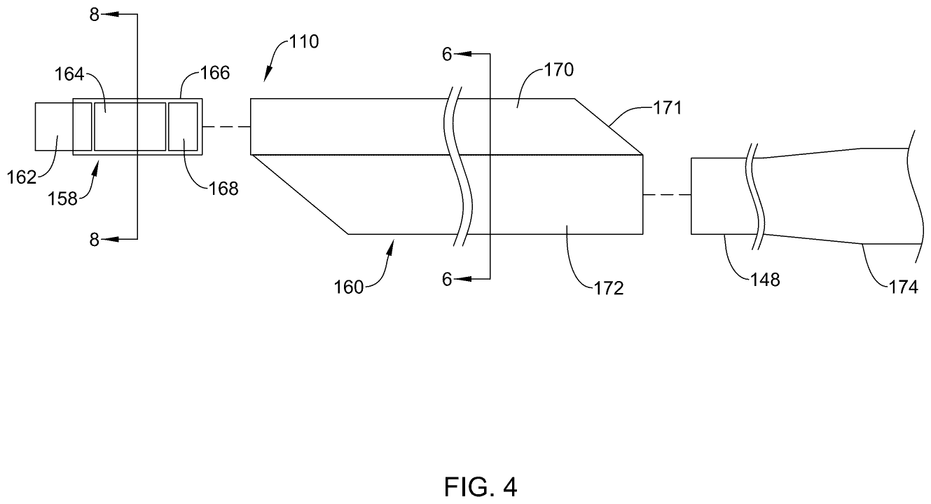

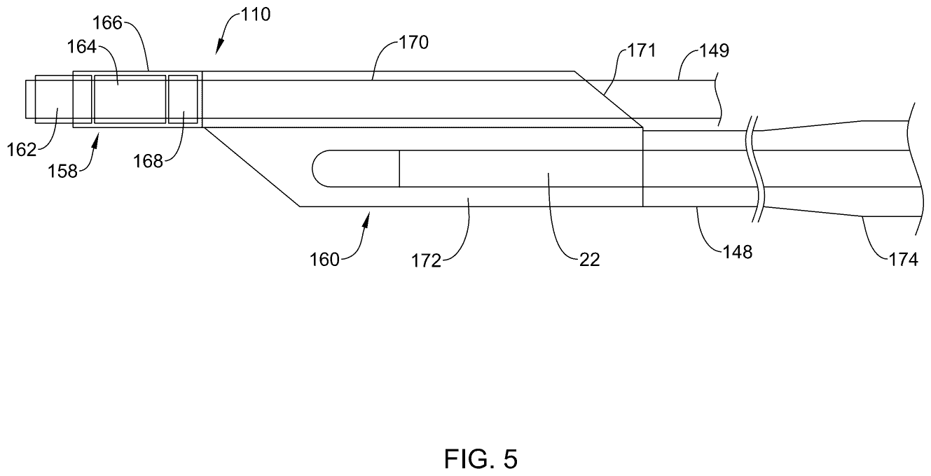

FIGS. 4-5 illustrate a portion of an example medical device 110 that may be similar in form and function to other medical devices disclosed herein. FIG. 4 shows the distal portion of the medical device 110 in an exploded configuration. Here it can be seen that the medical device 100 may include a distal tip region 158 and a dual lumen region 160. In general, the distal tip region 158 is coupled to or otherwise secured to the dual lumen region 160. The dual lumen region 160, in turn, is coupled to or otherwise secured to an imaging window region 148 (e.g., similar to other imaging window regions disclosed herein, which may also be coupled to a hypotube and/or other proximal structures of the medical device 110 in a manner similar to what is disclosed herein).

The distal tip region 158 may be formed as an assembly that includes a plurality of components that are joined together. For example, the distal tip region may include a tip or bumper tip member 162 and a radiopaque or marker member 164. The bumper tip member 162 may be generally configured to provide an atraumatic distal end to the medical device 110. In some instances, the bumper tip member 162 may take the form of a tubular structure made from a suitable material such as nylon, nylon-12, polyether block amide, combinations thereof, and/or other suitable materials including those materials disclosed herein. One example of a suitable material may be GRILFLEX ELG 6260. Other materials are contemplated.

The marker member 164 may be generally configured to provide a desirable level visualization (e.g., fluoroscopic visualization) for the medical device 110. For example, the marker member 164 may include a radiopaque material such as tantalum, tungsten, gold, platinum, bismuth, barium sulfate, combinations thereof, and/or the like. In some instances, the marker member 164 may include a polymer resin loaded with a radiopaque material. Some additional details and contemplated constructions for the marker member 164 are disclosed herein.

A sleeve 166 may be disposed over the bumper tip member 162 and the marker member 164. In general, the sleeve 166 may aid in forming the distal tip region 158 and/or in bonding/securing the distal tip region 158 to the dual lumen region 160. The sleeve 166 may be formed from a suitable material such as a polyether block amide. For example, the sleeve 166 may be formed from a 63D PEBAX material. Other materials are contemplated including those materials disclosed herein. In some instances, the sleeve 166 may help to improve the bond between the distal tip region 158 and the dual lumen region 160. In some of these and in other instances, the sleeve 166 may also help to cover, contain, and/or encapsulate the radiopaque materials within the marker member 164. For example, if the marker member 164 includes tungsten particles, the sleeve 166 can help to contain the tungsten particles within the distal tip region 158, which may help to reduce exposure of the tungsten particles to the patient.

In some instances, a spacer member 168 may be disposed adjacent to the marker member 164. For example, the spacer member 168 may be disposed between the proximal end of the marker member 164 and a distal end or distal end region of the dual lumen region 160. The spacer member 168 may provide a number of desirable features. For example, the spacer member 168 may aid in bonding the distal tip region 158 with the dual lumen region 160. In addition, the spacer member 168 may help provide a desirable level of tensile strength (e.g., at or near the bond between the distal tip region 158 and the dual lumen region 160). The spacer member 168 may be formed from a suitable material such as a polyether block amide. For example, the spacer member 168 may be formed from a 63D PEBAX material. Other materials are contemplated including those materials disclosed herein.

Manufacturing the distal tip region 158 may include disposing the tip member 162, the marker member 164, and the spacer member 168 (e.g., when included in the distal tip region 158) along a substrate or mandrel. The sleeve 166 may be disposed over the tip member 162, the marker member 164, and the spacer member 168 and the assembly may be thermally bonded. This may include the use of a hot jaw or other thermal bonding tool. In some instances, a heat shrink tube (not shown) may be disposed over the assembly (e.g., over the sleeve 166), which may help the bonding process, for example, by helping to maintain the position of the various structures during bonding (e.g., during thermal bonding). The heat shrink tube can be removed after forming the distal tip region 158. The assembled distal tip region 158 may bonded to the dual lumen region 160 using a similar thermal bonding process (e.g., which may include the use of a hot jaw or other thermal bonding tool). The bonding of the distal tip region 158 to the dual lumen region 160 may include the use of a heat shrink tube, which may be subsequently removed.

As disclosed herein, the imaging window region 148 may be coupled to the dual lumen region 160 (e.g., a proximal end or proximal end region thereof). This may include a butt joint and/or thermal bond. A number of bonding processes and/or bonds are contemplated. Some of the additional bonds/bonding processes are disclosed herein. In some instances, the imaging window region 148 may include a bumped-out region 174. The bumped-out region 174 may have an enlarged outer diameter (e.g., relative to other regions of the imaging window region 148). For example, the distal of the bumped-out region 174, the imaging window region 148 may have an outer diameter of about 0.034 inches (0.8636 mm) to 0.036 (0.9144 mm) +/−0.01 inches (0.254 mm) whereas the bumped-out region 174 may have an outer diameter of about 0.041 inches (1.0414 mm) +/−0.01 inches (0.254 mm). In some instances, the inner diameter of the bumped-out region 174 may be the same as the remainder of the imaging window region 148. In other instances, the bumped-out region 174 may have a different (e.g. enlarged) inner diameter relative to the remainder of the imaging window region 148.

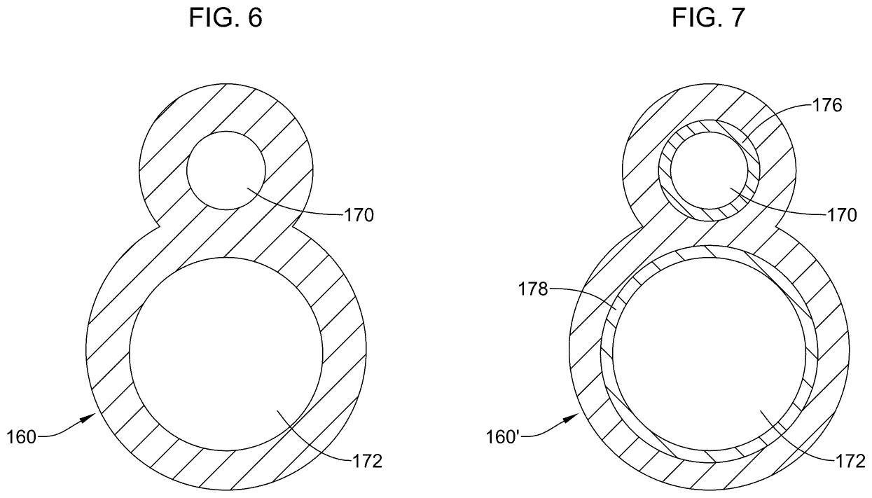

As can also be seen in FIG. 5, a guidewire 149 may be slidable disposed within a guidewire lumen 170 of the dual lumen region 160. In some instances, the dual lumen region 160 may include a skived or angled opening 171. This may aid in the insertion of the guidewire 149 therein. The imaging core 22 may be disposed in an imaging core lumen 172 of the dual lumen region 160. Turning now to FIG. 6, which is a cross-sectional view taken through line 6-6 in FIG. 4, the guidewire lumen 170 and the imaging core lumen 172 can be seen. In FIG. 6, the guidewire 149 and the imaging core 22 are removed. FIG. 7 is an alternative cross-sectional of a dual lumen region 160′. In this example, the dual lumen region 160′ may include an inner layer or liner 176 disposed along the guidewire lumen 170 and an inner layer or liner 178 disposed along the imaging core lumen 172. Suitable materials for the inner layers 176, 178 may include hydrophilic materials, ultra-high molecular weight (UHMW) polyethylene, high-density polyethylene (HDPE), a polyamide (e.g., such as polyamide 66 or PA66), a nylon (e.g., such as nylon 66), silicone, polytetrafluoroethylene (PTFE), ethylene tetrafluoroethylene (ETFE), polyetheretherketone (PEEK), polyimide (PI), acetal, polyethylene terephthalate (PET), fluorinated ethylene propylene (FEP), combinations thereof, and/or the like.

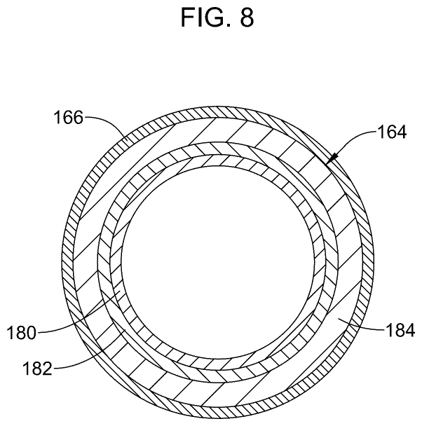

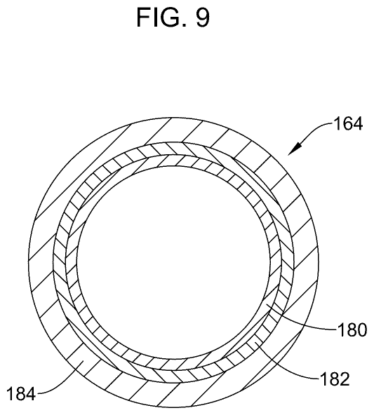

FIG. 8 is a cross-section view taken through line 8-8 in FIG. 4. Here the sleeve 166 can be seen disposed about the marker member 164. In addition, it can be seen that the marker member 164 may include a plurality of layers including a first or inner layer 180, a tie layer 182, and a second or outer layer 184. FIG. 9 illustrates the marker member 164 without the sleeve 166. In some instances, the inner layer 180 may include a polymer material such a polyethylene (e.g., a high-density polyethylene). This may provide a desirable level of lubricity and/or reduce friction along the marker member 164. In addition, the inner layer 180 may also help to may also help to cover, contain, and/or encapsulate the radiopaque materials within the marker member 164. In some of these and in other instances, the inner layer 180 may include an additive such as an ultra-high molecular weight polyethylene. Other materials are contemplated including those disclosed herein. The outer layer 184 may include a polymer resin loaded with a radiopaque material. This may allow for a desirable level of flexibility for the marker member 164 along with a desirable level of radiopacity. For example, the outer layer 184 may include a polyether block amide such as a 35D PEBAX material loaded with tungsten. In some of these and in other instances, the outer layer 184 may include an additive such as an ultra-high molecular weight polyethylene. Other materials are contemplated including those disclosed herein. The tie layer 182 may include a polyethylene (e.g., a low-density polyethylene). Other materials are contemplated including those disclosed herein.

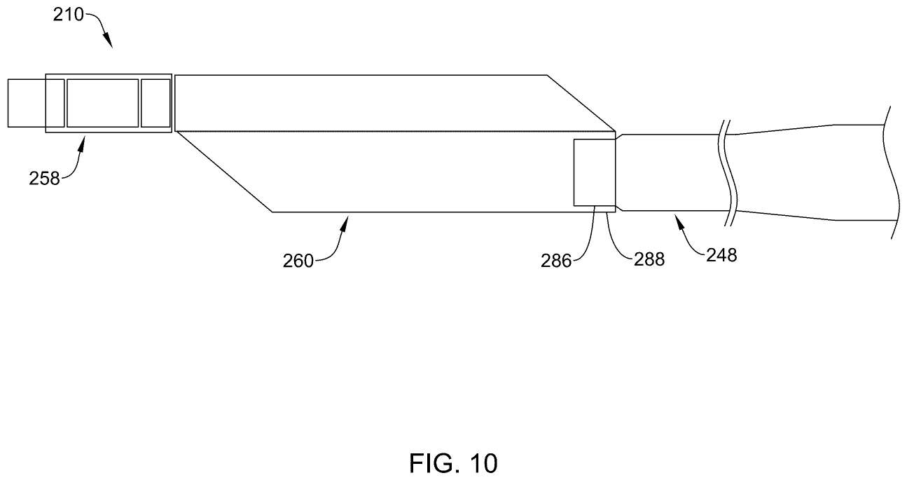

FIG. 10 illustrates an example medical device 210 that may be similar in form and function to other systems disclosed herein. The medical device 210 may include a distal tip region 258 and a dual lumen region 260. In this example, the imaging window region 248 may have a necked-down or reduced diameter region 286 that can be inserted into or fitted into (e.g., which may include an interference fit, adhesive bond, thermal bond, combinations thereof, etc.) the proximal end region 288 of the dual lumen region 260. In other words, a portion of the dual lumen region 260 may be disposed along an outer surface of the imaging window region 248. In some instances, the proximal end region 288 of the dual lumen region 260 may have an inner diameter of about 0.027 inches (about 0.6858 mm) +/−about 0.015 inches (+/−about 0.381 mm). The outer diameter of the reduced diameter region 286 may be slightly larger (e.g., about 1-10% larger, or about 2-6% larger, or about 4-5% larger) so that the reduced diameter region 286 can be inserted into the proximal end region 288 of the dual lumen region 260 and held by an interference fit. For example, outer diameter of the reduced diameter region 286 may be about 0.0283 inches (0.71882 mm) +/−about 0.015 inches (+/−about 0.381 mm). The reduced diameter region 286 may have a length of about 4-5 mm or so.

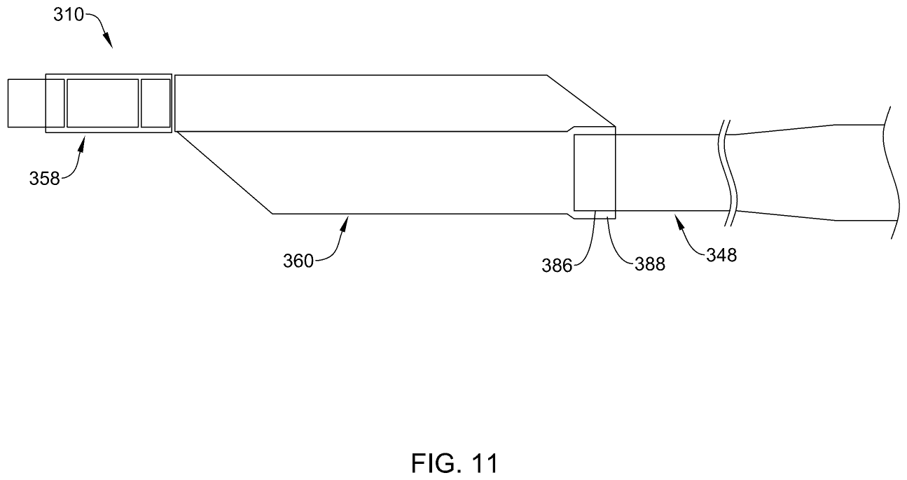

FIG. 11 illustrates an example medical device 310 that may be similar in form and function to other systems disclosed herein. The medical device 310 may include a distal tip region 358 and a dual lumen region 360. In this example, a proximal end region 388 of the dual lumen region 360 may flared or enlarged so that a distal end region 386 of the imaging window region 348 can be inserted into or fitted into (e.g., which may include an interference fit, adhesive bond, thermal bond, combinations thereof, etc.) the proximal end region 388 of the dual lumen region 360. In some instances, the proximal end region 388 of the dual lumen region 360 may have an inner diameter of about 0.0327 inches (about 0.83058 mm) +/−about 0.01 inches (+/−about 0.254 mm). The outer diameter of the distal end region 386 of the imaging window region 348 may be slightly larger (e.g., about 1-10% larger, or about 2-6% larger, or about 4-5% larger) than the inner diameter of the proximal end region 388 of the dual lumen region 360 so that the distal end region 386 of the imaging window region 348 can be inserted into the proximal end region 388 of the dual lumen region 360 and held by an interference fit. For example, the outer diameter of the distal end region 386 of the imaging window region 348 may be about 0.034 inches (0.8636) +/−about 0.01 inches (+/−about 0.254 mm).

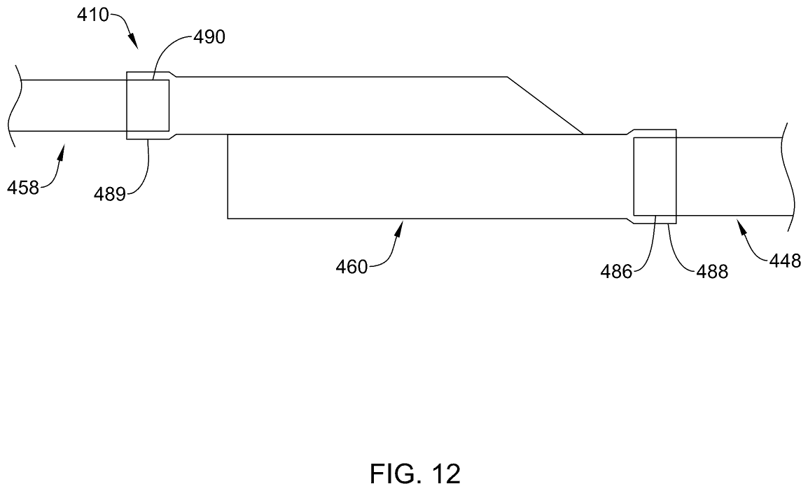

FIG. 12 illustrates an example medical device 410 that may be similar in form and function to other systems disclosed herein. The medical device 410 may include a distal tip region 458 and a dual lumen region 460. In this example, a proximal end region 488 of the dual lumen region 460 may flared or enlarged so that a distal end region 486 of the imaging window region 448 can be inserted into or fitted into (e.g., which may include an interference fit/bond, adhesive bond, thermal bond, combinations thereof, etc.) the proximal end region 488 of the dual lumen region 460. This is similar to what is shown and described in relation to FIG. 11. In addition, the dual lumen region 460 may include a flared distal end region 489 so that a proximal end region 490 of the distal tip region 458 can be inserted into or fitted into (e.g., which may include an interference fit/bond, adhesive bond, thermal bond, combinations thereof, etc.) the flared distal end region 489 of the dual lumen region 460.

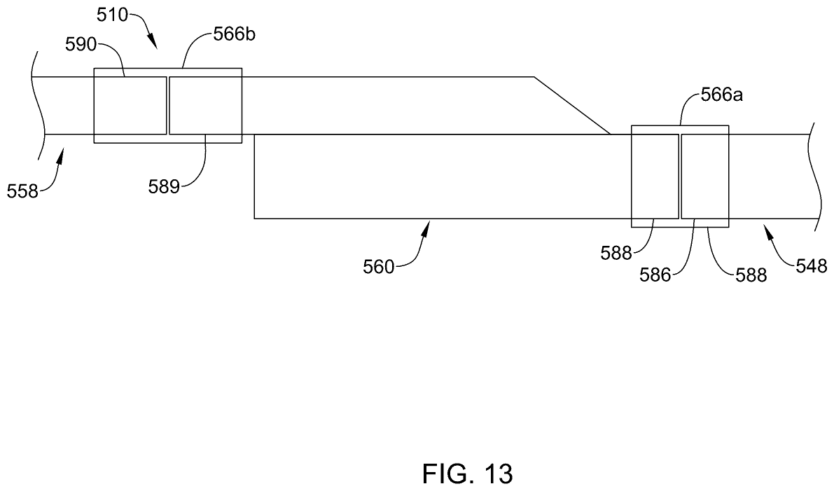

FIG. 13 illustrates an example medical device 510 that may be similar in form and function to other systems disclosed herein. The medical device 510 may include a distal tip region 558 and a dual lumen region 560. In this example, a proximal end region 588 of the dual lumen region 560 may be secured to a distal end region 586 of the imaging window region 548 with a sleeve 566a that is disposed over the end regions 586, 588. The sleeve 566a may be similar in form and function to other sleeves disclosed herein such as the sleeve 166. In some instances, a heat shrink tube (not shown) may be disposed over the assembly (e.g., over the sleeve 566a), which may help the bonding process, for example, by helping to maintain the position of the various structures during bonding (e.g., during thermal bonding). In addition, the dual lumen region 560 may include a distal end region 589 that may be secured to a proximal end region 590 of the distal tip region 558 with a sleeve 566b that is disposed over the end regions 589, 590. The sleeve 566b may be similar in form and function to other sleeves disclosed herein such as the sleeve 166.

The materials that can be used for the various components of the medical device 10 (and/or other devices disclosed herein) and the various tubular members disclosed herein may include those commonly associated with medical devices. For simplicity purposes, the following discussion refers to the elongate shaft 12 and other components of the medical device 10. However, this is not intended to limit the devices and methods described herein, as the discussion may be applied to other similar tubular members and/or components of tubular members or devices disclosed herein.

The elongate shaft 12 and/or other components of the medical device 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device 10 to achieve the same result.

In some embodiments, a degree of Magnetic Resonance Imaging (MRI) compatibility is imparted into the medical device 10. For example, the medical device 10, or portions thereof, may be made of a material that does not substantially distort the image and create substantial artifacts (e.g., gaps in the image). Certain ferromagnetic materials, for example, may not be suitable because they may create artifacts in an MRI image. The medical device 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An intravascular imaging device, comprising:
a catheter shaft including a proximal section and a distal section;
wherein the distal section includes a dual lumen region and a distal tip region coupled to the dual lumen region;
wherein the distal tip region includes a bumper tip member, a radiopaque member, and a sleeve disposed over and coupling the bumper tip member and the radiopaque member; and
an imaging core disposed within the catheter shaft.

2. The intravascular imaging device of claim 1, wherein the imaging core is translatable within the catheter shaft.

3. The intravascular imaging device of claim 1, wherein the imaging core includes an ultrasound transducer.

4. The intravascular imaging device of claim 1, wherein the imaging core includes an optical coherence tomography imaging device.

5. The intravascular imaging device of claim 1, wherein the dual lumen region has a distal end and further comprising a spacer member disposed between the radiopaque member and the distal end of the dual lumen region.

6. The intravascular imaging device of claim 5, wherein the sleeve extends over at least a portion of the spacer member.

7. The intravascular imaging device of claim 1, wherein the sleeve is thermally bonded to the dual lumen region.

8. The intravascular imaging device of claim 1, wherein the dual lumen region includes a guidewire lumen and an imaging core lumen.

9. The intravascular imaging device of claim 8, wherein the dual lumen region has a skived opening in fluid communication with the guidewire lumen.

10. The intravascular imaging device of claim 8, wherein an imaging window region is coupled to the dual lumen region adjacent to the imaging core lumen.

11. The intravascular imaging device of claim 10, wherein a portion of dual lumen region adjacent to the imaging core lumen is disposed along an outer surface of the imaging window region.

12. The intravascular imaging device of claim 10, wherein the imaging window region has a distal portion having a first outer diameter and a proximal portion having a second outer diameter larger than the first outer diameter.

13. An intravascular imaging device, comprising:
a catheter shaft including a proximal hypotube section, an imaging window section having a lumen formed therein, and a distal section;
wherein the distal section includes a dual lumen region and a distal tip region coupled to the dual lumen region;
wherein the dual lumen region defines a guidewire lumen and defines an imaging core lumen in fluid communication with the lumen formed in the imaging window section;
wherein the distal tip region includes a tip member, a radiopaque member, and a sleeve disposed over and coupling the tip member and the radiopaque member; and
an imaging core disposed within the catheter shaft.

14. The intravascular imaging device of claim 13, wherein the dual lumen region has a distal end and further comprising a spacer member disposed between the radiopaque member and the distal end of the dual lumen region.

15. The intravascular imaging device of claim 14, wherein the sleeve extends over at least a portion of the spacer member.

16. The intravascular imaging device of claim 13, wherein the sleeve is thermally bonded to the dual lumen region.

17. The intravascular imaging device of claim 13, wherein the dual lumen region has a skived opening in fluid communication with the guidewire lumen.

18. The intravascular imaging device of claim 13, wherein a portion of dual lumen region adjacent to the imaging core lumen is disposed along an outer surface of the imaging window section.

19. The intravascular imaging device of claim 13, wherein the imaging window section has a distal portion having a first outer diameter and a proximal portion having a second outer diameter larger than the first outer diameter.

20. A method for imaging a blood vessel, the method comprising:
disposing an intravascular imaging device within a blood vessel, the intravascular imaging device comprising:
a catheter shaft including a proximal section and a distal section,
wherein the distal section includes a dual lumen region and a distal tip region coupled to the dual lumen region,
wherein the distal tip region includes a bumper tip member, a radiopaque member, and a sleeve disposed over and coupling the bumper tip member and the radiopaque member, and
an imaging core disposed within the catheter shaft; and
translating the imaging core relative to the catheter shaft.
